(19) United States
(12) Patent Application Publication
(10) Pub. No.: US 2008/0131462 A1
(43) Pub. Date: Jun. 5, 2008

(54) LACTIC ACID BACTERIA STRAINS USEFUL AGAINST GASTROINTESTINAL PATHOGENS AND COMPOSITIONS CONTAINING SAME

(76) Inventors: Federico Graf, Zurich (CH); Philipp Grob, Zurich (CH); Dominique Brassart, Chavannes/Renens (CH)

Correspondence Address: WINSTON & STRAWN LLP;PATENT DEPARTMENT, 1700 K STREET, N.W., WASHINGTON, DC, 20006, US

(21) Appl. No.: 11/738,196

(22) Filed: Apr. 20, 2007

Related U.S. Application Data
(63) Continuation of application No. PCT/EP05/11151, filed on Oct. 17, 2005.

(30) Foreign Application Priority Data
Oct. 22, 2004 (EP) PCT/EP04/11981

(51) Int. Cl. A61K 39/07; A61K 35/74; A61P 31/04

(52) U.S. Cl. 424/246.1; 424/93.45

(57) ABSTRACT

The invention relates to a method for preventing or treating gastrointestinal infections in humans, which comprises administering a pharmaceutical preparation comprising, in combination with a pharmaceutically acceptable or food grade carrier, a therapeutically effective amount of at least one lactic acid bacteria strain of the genus L. acidophilus, L. crispatus, L. gasseri, L. helveticus and L. jensenii selected for their ability to kill urogenital and/or gastrointestinal pathogens and their ability to inhibit internalization of urogenital and/or gastrointestinal pathogens within gastrointestinal epithelial cells.

### CROSS-REFERENCE TO RELATED APPLICATIONS

This application is a continuation of international application PCT/EP2005/011151 filed Oct. 17, 2005, the entire content of which is expressly incorporated herein.

### FIELD OF THE INVENTION

This invention refers to the treatment of infectious troubles caused by various pathogens in humans, more specifically to the prevention and/or the treatment of gastrointestinal infections in humans.

### BACKGROUND OF THE INVENTION

Several experimental and clinical studies have assessed already the potential of certain lactobacilli in the prevention or treatment of certain gastro-intestinal tract infections and relevant therapy is applied for many decades already. Gastro-intestinal infections remain a common problem in the human population. Bacterial adherence to the gastrointestinal epithelium has been recognized as an important mechanism in the initiation and pathogenesis of gastrointestinal tract infections (GIT). Many gastrointestinal pathogens which colonize the intestinal tract may, depending on host factors and bacterial virulence factors, express virulence characteristics that enable them to resist the normally efficient host defense mechanisms.

The use of bacteria originating from the autochthonous micro flora, like e.g. lactobacilli, to exclude pathogens from colonizing the gastrointestinal tract is a concept which has been studied rather extensively (see e.g. Alain Servin in “Antagonist activities of lactobacilli and bifidobacteria against microbial pathogens—FENS Microbiology Reviews 2004). Some of the lactic acid bacteria strains mentioned in the above literature have been highlighted for their effect in the gastrointestinal tract and been proposed as possible active agents suitable for treating various troubles or disorders caused by pathogens, e.g. diarrhea.

Lactobacilli, when used in this context, are believed to contribute to the control of the local micro flora by different mechanisms such as pathogen growth inhibition, prevention of pathogens adherence, production of lactic acid and antagonistic substances like bacteriocins and H₂O₂, killing effect through said bacteriocins-like substances, immune-modulation, anti-inflammation and other mechanisms.

The main goal of therapy with bacterial agents should be to prevent overgrowth of pathogens until such a time that the normal intestinal micro flora can be re-established. In addition, bacterial therapy is considered as “natural” and without side effects in contrast with conventional chemical or pharmaceutical treatments. Within that context it has been surprisingly observed that lactic acid bacteria strains representative of the healthy human vaginal flora exhibited efficiency in the treatment of urogenital infections (see e.g. International Patent Application no PCT/EP2004/011980 filed on 5 Oct. 22, 2004 by Medinova AG, CH-Zurich) were also performing and consequently useful in the prophylactic or therapeutic treatment of intestinal infections or disorders initiated by gastrointestinal pathogens.

In addition thereto and despite of the progresses which have already been made concerning the knowledge of probiotic lactic acid bacteria (LAB) strains, their properties and their potential use in the pharmaceutical area, there still remained a need to propose more convenient and more efficient LAB strains to the medical community.

The purpose of this invention is to provide useful LAB strains and compositions particularly efficient in the treatment of infections caused by various pathogens, more specifically in the treatment of gastrointestinal infections in humans, or in the restoration of a balanced and healthy gastrointestinal flora after e.g. severe medical treatments like those performed with antibiotics. This invention provides as well new methods of prophylactic or therapeutic treatment of such infections which involve specifically selected LAB strains.

### SUMMARY OF THE INVENTION

The invention relates to a method for preventing or treating gastrointestinal infections in humans, which comprises administering a pharmaceutical preparation comprising, in combination with a pharmaceutically acceptable or food grade carrier, a therapeutically effective amount of at least one lactic acid bacteria strain of the genus L. acidophilus, L. crispatus, L. gasseri, L. helveticus and L. jensenii selected for their ability to kill urogenital and/or gastrointestinal pathogens and their ability to inhibit internalization of urogenital and/or gastrointestinal pathogens within gastrointestinal epithelial cells.

The invention also refers to a method for preventing or inhibiting adhesion, colonization and/or growth of pathogens in the gastrointestinal tract of humans, which comprises administering pharmaceutical preparation comprising, in combination with a pharmaceutically acceptable or food grade carrier, a therapeutically effective amount of at least one of said LAB strains.

The invention further refers to a method establishing, maintaining or restoring a healthy flora in the gastrointestinal tract of humans, which comprises administering pharmaceutical preparation comprising, in combination with a pharmaceutically acceptable or food grade carrier, a therapeutically effective amount of at least one of said LAB strains.

The invention also provides compositions, in particular pharmaceutical compositions useful within the above mentioned frame, which comprise a therapeutically effective amount of at least one LAB strain of the genus L. acidophilus, L. crispatus, L. gasseri, L. helveticus and L. jensenii selected for their ability to kill urogenital and/or gastrointestinal pathogens and then-ability to inhibit internalization of urogenital and/or gastrointestinal pathogens within gastrointestinal epithelial cells, in combination with a pharmaceutically acceptable carrier.

Eventually, this invention refers to the use of one of said LAB strains in the preparation of compositions, more particularly pharmaceutical compositions mentioned here above.

### DETAILED DESCRIPTION OF THE INVENTION

The LAB strains of this invention have been first selected for their ability to adhere to epithelial cells such as cervix HeLa or Caco-2 which were chosen as models. Cell adhesion is indeed a prerequisite selection feature as it conditions the capacity of the said LAB strains to colonize epithelial tissues, e.g. that of the urogenital tract, and then to compete with, inhibit or exclude pathogens adhesion from that specific location.

The said LAB strains have been further selected for their additional ability to inhibit adhesion, growth and even survival of pathogens, namely urogenital and gastrointestinal pathogens from epithelial cells. Gram-negative or Gram-positive pathogens such as those mentioned here after are representative of those which are significantly affected by the LAB strains of this invention in terms of adhesion, growth or pathogenic activity: Salmonella species like e.g. S. enterica serovar Typhimurium, E. coli and Staphylococcus species, e.g. S. aureus; this enumeration is of course not exhaustive.

The LAB strains of the present invention have also the ability to inhibit internalization of pathogens, namely urogenital or gastrointestinal Gram-negative or Gram-positive pathogens within epithelial cells.

The LAB strains of this invention, eventually, exhibit a further important feature, i.e. the ability to modulate the immune response of gastrointestinal mucous membrane cells in other words the ability to initiate, stimulate or reinforce the immune response of said cells when infected by gram-negative pathogens like those mentioned here above, in particular urogenital pathogenic E. coli. Due to their specific feature the said LAB strains have consequently the capacity to inhibit the inflammatory syndrome of the gastrointestinal mucous membrane cells when exposed to pathogen contamination.

Quite interestingly that specific feature performs the modulation of the immune response referred to here above using two distinct routes, i.e. via the induction of either pro- or anti-inflammatory cytokines like IL10, respectively, IL12, TNF or IFN. It has been further observed that some LAB strains of this invention exhibit a high IFNγ induction potential, namely L. acidophilus KS 116.1 and L. gasseri KS 124.3, a feature which favors the use of same as anti-infectious agents.

That strain specificity provides consequently to the man skilled in the art the possibility to select the most appropriate strain or combination of strains for performing the medical treatment which is looked for.

Among the LAB strains which exhibit these properties preferred species according to this invention are listed here after: L. jensenii KS 109, L. gasseri KS 114.1, L. crispatus KS 116.1, L. jensenii KS 119.1, L. crispatus KS 119.4, L. gasseri KS 120.1, L. jensenii KS 121.1, L. jensenii KS 122.1, L. gasseri KS 123.1, L. gasseri 124.3, L. gasseri KS 126.2, L. crispatus 127.1, L. jensenii KS 129.1, L. jensenii KS 130.1, L. helveticus KS 300 and L. acidophilus KS 400. Most of these strains are representative of the healthy human vaginal flora.

As particularly preferred species, one can further cite the following strains: L. gasseri KS 114.1 (CNCM1-3482): gram positive-catalase negative-oxidase negative-lactic acid production 10.5 g/l-H₂O₂ production 10 mg/l

API 50 CHI test: positive for GAL, GLU, FRU, MNE, NAG, ESC, SAL, CEL, MAL, SAC, TRE and GEN

negative for: KON, GLY, ERY, DARA, LARA, RIB, DXYL, LXYL, ADO, MDX, SBE, RHA, DUL, INO, MAN, SOR, MDM, MDG, AMY, ARE, LAC, MEL, INU, MLZ, RAF, AMD, GLYG, XLT, TUR, LYX, TAG, DFUC, LFUC, DARL, LARL, GNT, 2KG and 5KG

L. crispatus KS 116.1 (CNCM 1-3483): gram positive-catalase negative-oxidase positive-lactic acid production 9.6 g/l-H₂O₂ production 2 mg/l

API 50 CHI test: positive for GAL, FRU, MNE, NAG, ESC, SAL, MAL and SAC

negative for: KON, GLY, ERY, DARA, LARA, RIB, DXYL, LXYL, ADO, MDX, GAL, SBE, RHA, DUL, INO, MAN, SOR, MDM, MDG, AMY, ARB, CEL, LAC, MEL, TRE, INU, MLZ, RAF, AMD, GLYG, XLT, GEN, TUR, LYX, TAG, DFUC, LFUC, DARL, LARL, GNT, 2KG and 5KG

L. jensenii KS 119.1 (CNCM 1-3217): gram positive-catalase negative-oxidase negative-lactic acid production 7.4 g/l-H₂O₂ production 20 mg/l

API 50 CHI test: positive for GLU, FRU, MNE, NAG, AMY, ESC, SAL, CEL, MAL, MEL, SAC, GEN and TAG—variable for: RIB

negative for: KON, GLY, ERY, DARA, LARA, DXYL, LXYL, ADO, MDX, GAL, SBE, RHA, DUL, INO, MAN, SOR, MDM, MDG, ARB, LAC, TRE, INU, MLZ, RAP, AMD, GLYG, XLT, TUR, LYX, DFUC, LFUC, DARL, LARL, GNT, 2KG and 5KG

L. crispatus KS 119.4 (CNCM 1-3484): gram positive-catalase negative-oxidase positive-lactic acid production 10.3 g/l-H₂O₂ production negative

API 50 CHI test: positive for GAL, GLU, FRU, MNE, NAG, ESC, MAL, LAC, SAC and AMD

negative for: KON, GLY, ERY, DARA, LARA, RIB, DXYL, LXYL, ADO, MDX, SBE, RHA, DUL, INO, MAN, SOR, MDM, MDG, AMY, ARB, SAL, CEL, MEL, TRE, INU, MLZ, RAF, GLYG, XLT, GEN, TUR, LYX, TAG, DFUC, LFUC, DARL, LARL, GNT, 2KG and 5KG

L. gasseri KS 120.1 (CNCM 1-3218): gram positive-catalase negative-oxidase negative-lactic acid production 10.6 g/l-H₂O₂ production 1 mg/l

API 50 CHI test: positive for: GAL, GLU, FRU, MNE, AMY, ESC, SAL, CEL, MAL, LAC, SAC, THE and AMD

negative for: KON, GLY, ERY, DARA, LARA, RIB, DXYL, LXYL, ADO, MDX, SBE, RHA, DDL, INO, MAN, SOR, MDM, MDG, NAG, ARE, MEL, INU, MLZ, RAF, GLYG, XLT, GEN, TUR, LYX, TAG, DFUC, LFUC, DARL, LARL, GNT, 2KG and 5KG

L. jensenii KS 121.1 (CNCM1-3219): gram positive-catalase negative-oxidase negative-lactic acid production 10.6 g/l-H₂O₂ production 1 mg/l

API 50 CHI test: positive for: GAL, GLU, FRU, MNE, AMY, ARE, ESC, SAL, CEL, MAL, SAC and AMD—variable for: RIB, NAG, LAC, RAF and LFUC

negative for: KON, GLY, ERY, DARA, LARA, DXYL, LXYL, ADO, MDX, SBE, RHA, DUL, INO, MAN, SOR, MDM, MDG, MEL, TRE, INU, MLZ, GLYG, XLT, GEN, TUR, LYX, TAG, DFUC, DARL, LARL, GNT, 2KG and 5KG

L. gasseri KS 123.1 (CNCM 1-3485): gram positive-catalase negative-oxidase negative-lactic acid production 8.5 g/l-H₂O₂ production 10 mg/l

API 50 CHI test: positive for: GLU, MNE, NAG, ESC, MAL and SAC—variable for RIB and 5KG

negative for: KON, GLY, ERY, DARA, LARA, DXYL, LXYL, ADO, MDX, GAL, FRU, SBE, RHA, DUL, INO, MAN, SOR, MDM, MDG, AMY, ARB, SAL, CEL, LAC, MEL, TRE, INU, MLZ, RAF, AMD, GLYG, XLT, GEN, TUR, LYX, TAG, DFUC, LFUC, DARL, LARL, GNT and 2KG

L. gasseri KS 124.3 (CNCM 1-3220): gram positive-catalase negative-oxidase negative-lactic acid production 17.0 g/l-H₂O₂ production 20 mg/l

API 50 CHI test: positive for: GAL, GLU, FRU, MNE, NAG, ESC, SAL, MAL, SAC and TRE—variable for: RIB, AMD, GEN and 5KG

negative for: KON, GLY, ERY, DARA, LARA, DXYL, LXYL, ADO, MDX, SBE, RHA, DUL, INO, MAN, SOR, MDM, MDG, AMY, ARB, CEL, LAC, MEL, INU, MLZ, RAF, GLYG, XLT, TUR, LYX, TAG, DFUC, LFUC, DARL, LARL, GNT and 2KG

L. crispatus KS 127.1 (CNCM 1-3486): gram positive-catalase negative-oxidase positive-lactic acid production 16.7 g/l-H₂O₂ production negative

API 50 CHI test: positive for RIB, GAL, GLU, FRU, MNE, MAN, SOR, NAG, AMY, ESC, SAL, CEL, MAL LAC, SAC, TRE, MLZ, AMD, GLYG, GEN, TAG and GNT—variable for GLY and DXYL

negative for: KON, ERY, DARA, LARA, LXYL, ADO, MDX, SBE, RHA, DUL, INO, MDM, MDG, ARB, MEL, INU, MLZ, RAF, XLT, TUR, LYX, DFUC, LFUC, DARL, LARL, 2KG and 5KG

L. helveticus KS 300 (CNCM1-3360): gram positive-lactic acid production 10.45 g/kg H₂O₂ production 1 mg/l

API 50 CHI test-positive for: GAL, GLU, FRU, MNE, AMY, ARE, ESC, SAL, GEL, MAL, LAC, SAC, TRE and AMD

negative for: RIB, MAN, GLY, SOR, KON, ERY, MLZ, DARA, LARA, LXYL, ADO, MDX, SBE, RHA, DUL, INO, MDM, MDG, MEL, INU, RAF, TAG, GNT, XLT, TUR, LYX, DFUC, LFUC, DARL, LARL, 2KG and 5KG.

These strains have been duly registered at the Pasteur Institute, Paris (France) in accordance with the Budapest Treaty.

According to the present invention, due to their specific antipathogen activity, the LAB strains can be used advantageously for preventing or treating gastrointestinal infections in humans and for preventing or inhibiting the colonization and/or growth of pathogens in the gastrointestinal tract of humans as well, i.e. in a context wherein said LAB strains proved particularly efficient.

Also, the said LAB strains can be used in a quite efficient way for maintaining or restoring a healthy gastrointestinal flora in humans, more particularly for restoring a balanced and healthy gastrointestinal flora after severe medical treatments like those performed with antibiotics.

The corresponding therapeutic or prophylactic treatments are performed by administering a therapeutically effective amount of LAB strains of this invention in combination with a pharmaceutically acceptable or food grade excipient, support or carrier which has been designed therefor.

Compositions suitable to perform the above treatments can further comprise the usual LAB growth factors. Said compositions are preferably in the form of ingestible capsules or gelules comprising lyophilized microorganisms and LAB growth factors if ever required, in the form of edible suspensions or emulsions.

Compositions as those mentioned here above can comprise mixtures of LAB strains of this invention and mixtures of at least one of these strains together with one or several strains of the prior art as well. Also, these compositions may contain additional pharmaceutically active ingredients like chemical compounds specifically selected.

The compositions referred to here above may contain the selected microorganisms in amounts which can range from about 10⁶ cfu (colony forming units), preferably from about 10⁸ to about 10¹¹ cfu per g or dose or unit, usually in a dehydrated form that keeps their viability and their specificity intact, e.g. in a lyophilized form. The ultimate details of said compositions as well as their dosage shall depend eventually on the specific application they are intend for, the age or health status of the patients, the nature of the pathogen contamination. It is within current skills and expertise of the medical community to adjust all the relevant parameters.

When compared to prior known reference strains (see examples below) the LAB strains of the present invention have shown either similar or higher antipathogens activity depending on the experimental model which has been selected therefor.

The following examples illustrate only some of the embodiments of this invention and so are not intended to constitute any limitation or restriction thereof.

### EXAMPLES

Material and Methods

| Tested Strain | Code |
|---|---|
| L. jensenii 109 | KS 109 |
| L. crispatus 116.1 | KS 116.1 |
| L. jensenii 119.1 | KS 119.1 |
| L. gasseri 120.1 | KS 120.1 |
| L. jensenii 121.1 | KS 121.1 |
| L. jensenii 122.1 | KS 122.1 |
| L. gasseri 124.3 | KS 124.3 |
| L. gasseri 126.2 | KS 126.2 |
| L. jensenii 129.1 | KS 129.1 |
| L. jensenii 130.1 | KS 130.1 |
| L. helveticus 300 | KS 300 |
| L. acidophilus 400 | KS 400 |

The control adhering lactobacilli strain are the L. casei rhamnosus strain GG (ATCC Accession no 53103), the L. rhamnosus strain GR-1 (ATCC Accession no 55826) and the L. fermentum strain RC-14 (ATCC Accession no 55845).

All the lactobacilli strains were grown in De Man, Rogosa, Sharpe (MRS) broth (Biokar Diagnostic, Beauvais, France) for 18 h at 37° C.

Bacterial pathogens. Salmonella enterica serovar Typhimurium strain SL 1344 was a gift of B.A.D. Stocker (Stanford, Calif.). Bacteria were grown overnight for 18 h at 37° C. in Luria broth (Difco Laboratories).

Uropathogenic diffusely-adhering Escherichia coli strains IH11128 and 7372, and diarrheagenic strain C1845 were gifts from B. Nowicki (Texas University, Galvestone) and S. Moseley (Seattle University). Strain 7372 carries the class II papG allele, the hly gene (haemolysin) and the Dr operon. Strain IH11128 carries the Dr operon. Strain C1845 carries the Daa operon. All bacterial strains were maintained on LB plates and prior to infection, bacteria were grown in LB broth at 37° C. for 18 h.

Staphylococcus aureus strain was from the Pasteur culture collection (Paris). Bacteria were grown overnight at 37° C. in TSA broth (Difco Laboratories).

Bacteria were suspended in buffered sodium chloride-peptone solution pH 7.0 to about 10⁶ colony forming unit (CFU/ml). 500 ul or the prepared suspensions were spread out on the agar plate. The inoculated plates were dried under sterile laminar air flow conditions. The agar plates were then incubated under anaerobic conditions using a sealed anaerobic jar (Becton Dickinson, USA) at 37° C. for 36 h in maximum. Before use, the Gardnerella vaginalis strain was sub-cultured in BHI supplemented with yeast extract, maltose and horse serum, under anaerobic conditions using a sealed anaerobic jar at 37° C. for 36 h in maximum.

Before use, bacterial cultures were centrifuged at 5.500×g for 5 mm at 4° C. The culture medium was discarded and the bacteria were washed once with phosphate-buffered saline (PBS) and re suspended in PBS.

Cell lines and cultures. Human cervical HeLa cells were cultured at 37° C. in a 5% CO₂-95% air atmosphere in RPMI 1640 with L-glutamine (Life Technologies) supplemented with 10% heat-inactivated (30 min, 56° C.) foetal calf serum (FCS; Boehringer, Mannheim, Germany), as previously described. Cells were used for infection assays before confluence, i.e., after 5 days in culture.

The human intestinal cell line used was the TC7 clone (Caco-2/TC7), established from the parental Caco-2 cell line. Cells were routinely grown in Dulbecco modified Eagle's minimal essential medium (DMEM) (25 mM glucose) (Invitrogen, Cergy, France), supplemented with 15% heat-inactivated (30 min, 56° C.) foetal calf serum (Invitrogene) and 1% non-essential amino acids (Invitrogene) as previously described. For maintenance purposes, cells were passaged weekly using 0.02% trypsin in Ca²⁺Mg²⁺-free PBS containing 3 mM EDTA. Experiments and maintenance of the cells were carried out at 37° C. in a 10% CO₂/90% air atmosphere. The culture medium was changed daily. Cells were used at post-confluence after 15 days of culture (fully differentiated cells) for infection assay of S. enterica serovar Typhimurium.

Adhesion assays. The adhesion of lactobacilli strains onto cervix HeLa cells and intestinal Caco-2/TC7 cells was examined according to the following steps: the cells monolayers were washed twice with phosphate-buffered saline (PBS). For each adhesion assay, 0.5 ml of the Lactobacillus suspension (bacteria with spent broth culture supernatant) was mixed with DMEM (0.5 ml), and then added to each well of the tissue culture plate (24 wells) which was then incubated at 37° C. in 10% CO₂/90% air. The final concentrations of bacteria examined were 1×10⁸, 2×10⁸, 1×10⁹, and 2×10⁹ bacteria per ml. After 1 h incubation, the monolayers were washed five times with sterile PBS, fixed with methanol, stained with Gram stain, and then examined microscopically under oil immersion. Each adhesion assay was conducted in duplicate with cells from three successive passages. For each assay, the number of adherent bacteria was determined in 20 random microscopic areas (adhesion score: 0 to 5). Moreover, the level of viable adhering lactobacilli was determined by quantitative determination of bacteria associated with the infected cell monolayers. After being infected, cells were washed twice with sterile PBS and lysed with sterilized H₂O. Appropriate dilutions were plated on tryptic soy agar (TSA) to determine the number of viable cell-associated bacteria by bacterial colony counts.

Each cell-association assay was conducted at least in triplicate, with three successive cell passages. Results were expressed as CPU/ml of cell-associated bacteria.

Activity against the growth of pathogens. A culture medium containing MRS (5 ml) and specific pathogen culture medium (5 ml) was inoculated with 0.1 ml of a cultivated pathogen and 0.1 ml of cultured Lactobacillus strain. Control was a culture medium inoculated with 0.1 ml of a cultivated pathogen and 0.1 ml of non-cultivated MRS adjusted to pH 4.5. At indicated time-points, aliquots were removed, serially diluted and plated on tryptic soy agar to determine bacterial colony counts of pathogen. Each assay was conducted at least in triplicate. Results were expressed as CFU/ml.

Activity against the viability of pathogens. Colony count assays were performed by incubating 10⁸ CFU/ml pathogen (0.5 ml) with the lactobacilli culture (10⁸ CFU/ml, 0.5 ml) at 37° C. Control was non-cultivated MRS adjusted to pH 4.5. Initially and at predetermined 15 intervals, aliquots were removed, serially diluted and plated on tryptic soy agar to determine bacterial colony counts of pathogen. Each assay was conducted at least in triplicate. Results were expressed as CFU/ml.

Inhibition of uropathogenic E. coli adhesion onto epithelial HeLa cells. For cell monolayer infection, pathogens were cultured at 37° C. for 18 h in appropriate culture media as described above. Prior to infection, the cell monolayers, prepared in twenty four-well TPP tissue culture plates (ATGC, Paris, France), were washed twice with PBS. Infecting bacteria were suspended in the culture medium and a total of 0.5 ml DMEM+0.25 ml culture pathogen (1×10⁸ CFU/ml)+0.25 ml lactobacilli culture (1.5×10⁹ CFU/ml) were added to each well of the tissue culture plate. The plates were incubated at 37° C. in 10% CO₂/90% air for different time of infection as indicated and then were washed three times with sterile PBS and lysed with sterilized H₂O. Appropriate dilutions were plated on tryptic soy agar to determine the number of viable cell-associated bacteria by bacterial colony counts. Each assay was conducted hi triplicate with three successive passages of HeLa cells.

Analysis. Results are expressed as means ±standard error to the mean. For statistical comparisons, Student's t test was performed.

### Results

### Example 1

### 1. Adhesion Capacity of L. jensenii KS 119.1 and KS 130.1, L. crispatus KS 116.1 and L. gasseri KS 124.3 onto HeLa and Caco-2/TC7 Cells

The level of adhesion of the above strains was determined after the cells were inoculated with four concentrations of lactobacilli (5×10⁷; 1×10⁸; 5×10⁸; 1×10⁹ CPU/well). Generally, a concentration-dependent adhesion was observed.

In cervix HeLa cells, adhesion levels observed show that all the tested strains are adhering. The L. jensenii KS 119.1 and KS 130.1 strains appeared the best adhering strains (7.5 log CFU/ml at 5×10⁸ CPU/well) as compared with the control adhering strains, L. casei rhamnosus GG and L. rhamnosus GR1 strains.

In intestinal Caco-2/TC7 cells, adhesion levels observed show that all the Medinova strains are adhering. The L. crispatus KS 116.1, L. jensenii 119.1, 129.1 and KS 130.1, L. gasseri 124.3 strains appeared the best adhering strains (7.5-8 logs CFU/ml at 5×10⁸ CPU/well) as compared with the control adhering strains, L. casei rhamnosus GG and L. rhamnosus GR1 strains.

As observed by scanning electron microscopy, all the “invention lactobacilli strains” appeared adhering in close contact with the HeLa and Caco-2/TC7 cells.

On the basis of their adhering properties, the L. crispatus KS 116.1 and L. jensenii 119.1 have been selected for the following studies of antibacterial activities against urovaginal and intestinal pathogens.

### 2. Activity of KS 116.1 and KS 119.1 on the Growth of Urogenital and Intestinal Pathogens

It has been examined whether the above mentioned strains are active on the growth of Staphylococcus aureus, uropathogenic and diarrheagenic E. coli, and diarrheagenic Salmonella enterica serovar Typhimurium. The growth of pathogens was measured at 5, 8, 18 and 24 h.

For Staphylococcus aureus, the control L. rhamnosus strain GR-1 and L. fermentum strain RC-14 inhibited the growth of bacteria. Similarly, L. crispatus KS 116.1 and L. jensenii KS 119.1 inhibited the growth of Staphylococcus aureus and showed a decrease in the viable bacteria number. When activities of lactobacilli strains were compared, the L. jensenii KS 119.1 appeared the most active strain.

For uropathogenic E. coli strains IH11128 and 7372, the control L. rhamnosus strain GR-1 and L. fermentum strain RC-14 inhibited the growth of the bacteria. Similarly, L. crispatus KS 116.1 and L. jensenii KS 119.1 inhibited the growth of E. coli. When activities of lactobacilli strains were compared, the L. jensetrii 119.1 appeared the most active strain.

For diarrheagenic E. coli strain C1845, the control L. rhamnosus strain GR-1 and L. fermentum strain RC-14 inhibited the growth of the bacteria. Similarly, L. crispatus KS 116.1 and L. jensenii KS 119.1 inhibited the growth of E. coli. When activities of lactobacilli strains were compared, the same activity was found for all the lactobacilli strains examined.

For diarrheagenic S. enterica serovar Typhimurium strain SL1344, the control L. rhamnosus strain GR-1 and L. fermentum strain RC-14 inhibited the growth of the bacteria. Similarly, L. jensenii 119.1 inhibited the growth of S. enterica serovar Typhimurium. When activities of lactobacilli strains were compared, the same activity was found for the control L. rhamnosus strain GR-1 and L. fermentum strain RC-14 and L. jensenii KS 119.1. In contrast, the L. crispatus KS 116.1 showed a lower activity.

### 3. 11 3. Killing Activity of KS 116.1 and KS 119.1 against Urogenital and Intestinal Pathogens

It has been examined whether said lactobacilli are active on the viability of Staphylococcus aureus, uropathogenic and diarrheagenic E. coli, and diarrheagenic Salmonella enterica serovar Typhimurium. The effect on viability of pathogens was measured at 2, 3, and 4 h.

For Staphylococcus aureus, the control L. rhamnosus strain GR-1 and L. fermentum strain RC-14, and L. jensenii KS 119.1 decreased for 2 logs the viability of bacteria. In contrast, the L. crispatus KS 116.1 showed no activity.

For uropathogenic E. coli strains IH11128 and 7372, the control L. rhamnosus strain GR-1 and L. fermentum strain RC-14 showed 4 logs of decrease in viability of bacteria. L. crispatus KS 116.1 and L. jensenii KS 119.1 were not active showing only one log of decrease in viability of the bacteria.

For diarrheagenic E. coli strain C1845, both of the control L. rhamnosus strain GR-1 and L. fermentum strain RC-14, and L. crispatus KS 116.1 and, L. jensenii KS 119.1 showed a low activity on the viability of C1845 bacteria (2 logs of decrease).

For diarrheagenic S. enterica serovar Typhimurium strain SL1344, both of the control L. rhamnosus strain GR-1 and L. fermentum strain RC-14, and L. crispatus KS 116.1 and L. jensenii KS 119.1 showed a great activity by decreasing the viability of SL1344 bacteria (5 logs of decrease).

### 4. Inhibition of the Adhesion of Uropathogenic E. coli Strain IH11128 Strain onto HeLa Cells by KS 116.1 and KS 119.1

It has been examined whether said lactobacilli are able to inhibit the adhesion of uropathogenic E. coli strain IH11128 onto HeLa cells. The effect of the control L. rhamnosus strain GR-1 and L. fermentum strain. RC-14, and L. jensenii 119.1 and L. crispatus KS 116.1 was measured at three concentrations: 1×10⁸, 5×10⁸ and 1×10⁹ bacteria per well.

A 30 to 40% of inhibition of IH11128 adhesion was found at a concentration of 1×10⁸ bacteria per well for the control L. rhamnosus strain GR-1 and L. fermentum strain RC-14. At this concentration, the L. jensenii KS 119.1 and L. crispatus KS 116.1 were inactive. Inhibition of IH11128 adhesion was effective at a concentration of 5×10⁸ bacteria per well for L. jensenii KS 119.1 and L. crispatus KS 116.1 and a similar inhibition that those obtained with the control L. rhamnosus strain GR-1 and L. fermentum strain RC-14 was observed. A similar high inhibition level of IH11128 adhesion was observed with the control L. rhamnosus strain GR-1 and L. fermentum strain RC-14, and L. jensenii KS 119.1 and L. crispatus KS 116.1 at the concentration of 1×10⁹ bacteria per well.

### Example 2

### 1. Activity of L. gasseri KS 124.3, L. helveticus KS 300 and L. acidophilus KS 400 on the Growth of Urogenital and Intestinal Pathogens

It has been examined whether the strains referred to here above are active against the growth of Staphylococcus aureus and uropathogenic and diarrbeagenic E. coli strains IH11128 and 7372. The growth of pathogens was measured at 5, 8, 18 and 24 h.

Concerning Staphylococcus aureus, the control L. rhamnosus strain GR-1 and L. fermentum strain RC-14 efficiently inhibited the growth of the bacteria. Similarly, L. gasseri KS 124.3, L. helveticus KS 300 and L. acidophilus KS 400 inhibited the growth of Staphylococcus aureus and showed a decrease in the viable bacteria number. When activities of lactobacilli strains were compared, the L. helveticus KS 300 appeared the most active strain.

For uropathogenic E. coli strains IH11128, the control strains L. rhamnosus GR-1 and L. fermentum RC-14 efficiently inhibited the growth of the bacteria. Similarly, L. helveticus KS 300 efficiently inhibited the growth of E. coli. When activities of lactobacilli strains were compared, a lower activity appeared for L. gasseri KS 124.3 and L. acidophilus KS 400.

For uropathogenic E. coli strain 7372, both control strains L. rhamnosus GR-1 and L. fermentum RC-14 strains inhibited the growth of bacteria. Similarly L. helveticus KS 300 inhibited the growth of said bacteria whereas L. acidophilus KS 400, however, was active only at 25 hours.

### 2. Killing Activity of KS 124.3, KS 300 and KS 400 against Urogenital and Intestinal Pathogens

It has been examined whether said lactobacilli are active on the viability of Staphylococcus aureus, uropathogenic E. coli IH11128 and 7372, and diarrheagenic E. coli C1845. The effect on viability of pathogens was measured at 2, 3, and 4 h.

For Staphylococcus aureus, the control strains L. rhamnosus GR-1 and L. fermentum RC-14, and L. gasseri KS 124.3, L. helveticus KS 300 and L. acidophilus KS 400 decreased for 2-3 logs the viability of bacteria.

For uropathogenic E. coli strains IH11128, the control strains L. rhamnosus strain GR-1 and L. fermentum RC-14 and L. helveticus KS 300 as well showed 3 logs of decrease in viability of the bacteria. L. acidophilus KS 400 and L. gasseri KS 124.3 were not active.

Concerning uropathogenic E. coli strains 7372, the control strains showed 2 logs of decrease in viability of the bacteria. L. helveticus KS 300 showed 3 logs of decrease whereas L. acidophilus KS 400 and L. gasseri KS 124.3 were not active within the same conditions.

For diarrheagenic E. coli strain C1845, both of the control strains L. rhamnosus GR-1 and L. fermentum RC-14 killed the bacteria showing a 3 log decrease in the viability of same. Similar effect was observed for L. gasseri KS 124.3 whereas no activity was detected concerning L. acidophilus KS 400. L. helveticus KS 300 exhibits a killing which is definitely higher that that observed for the above control strains.

### Example 3

### 1. Killing Activity of L. jensenii KS 121.1 and KS 122.1, L. gasseri KS 120.1 and L. helveticus KS 300 against Urogenital and Intestinal Pathogens

It has been examined whether said lactobacillus strains are active on the viability of uropathogenic E. coli IH11128 and Salmonella enterica Typhimurium. The effect on viability of pathogens was measured at 4 h of contact.

For uropathogenic E. coli strains IH11128, L. jensenii KS 121.1 and KS 122.1 showed no activity whereas, in contrast, L. gasseri KS 120.1 decreased efficiently (4 logs) the viability of E. coli in unshaken conditions. L. helveticus KS 300 and the L. fermentum RC-1 control strain decreased of 2 logs the viability of E. coli in unshaken conditions only.

Concerning Salmonella Typhimurium, L. gasseri KS 120.1 (3 logs), L. jensenii KS 121.1 and KS 122.1, L. helveticus KS 300 and the control strain L. fermentum RC-14 were quite active (6 logs of decrease) in unshaken conditions. L. gasseri KS 120.1 remained active even in shaken conditions.

### 2. Inhibition of Adhesion and Internalization of Uropathogenic E. coli Strain IH11128 Strain onto HeLa Cells by KS 120.1, KS 121.1 and KS 300

A strategy often used by extra-intestinal pathogens like E. coli to evade host defense mechanism is to establish a local reservoir within epithelial cells (M. A. Muvlea in Eschrichia coli. Cell. Microbiol. 4, 257-271—2002) and cell entry by IH11128 strain appears to be an effective mechanism for promoting prolonged persistence of these pathogens in the urinary tract.

The effect of L. gasseri KS 120.1, L. helveticus KS 300 and of the control strains RC-14 and GG strain was examined concerning the above uropathogenic E. coli: L. jensenii 121.1 decreased for 2 logs the level of viable internalized E. coli, whereas L. gasseri 120.1, L. helveticus KS 300 and both the control strains have shown a 4 logs of decrease of the internalized E. coli.

### Example 4

### Modulation of the Immune Response

### In Vivo Test Using Human PMBC

The following strains have been tested within the conditions set hereafter concerning their ability to induce or modulate or affect an immune response, more specifically their ability to induce the secretion of cytokines and the like: L. crispatus KS 116.1, L. jensenii 119.1, L. jensenii KS 121.1 and KS 122.1, L. gasseri KS 120.1, L. gasseri KS 124.3, L. helveticus KS 300 and L. acidophilus KS 400.

The detection of the induction of cytokines was made by means of a test for in vitro stimulation of isolated peripheral blood mononuclear cells (PBMC). Among the cytokines induced during these tests, there are interleukins 10 and 12 (IL10 & IL12), γ-interferon (Y—IFN) and tumor necrosis factor α (TNFα).

### Experimental Procedures

PMBC preparation: Fresh human blood obtained for healthy subjects (four donors) was diluted at a 1:2 ratio with PBS-Ca (GIBCO) and purified on a Ficoll gradient (GIBCO). After 5 centrifugation at 400×g for 30 min at 20° C. the peripheral blood monocular cellular cells (PMBC's) formed an interphase ring layer in the serum. PMBC's were aspired carefully, suspended to a final volume of 50 ml using PBD-Ca and washed three times in the same buffer with centrifugation steps at 350×g for 10 min at 20° C.

PMBC's were subsequently resuspended using complete RPMI medium (GIBCOP), supplemented with 10% w/v L-glutamine (GIBCO) and gentamycin (150 jig/ml) (GIBCO). PBMC's were counted under the microscope and adjusted at a concentration of 2×10⁶ cells/ml and distributed (in 1 ml aliquots) in 24-well tissues culture plates (Corning, Inc.).

Bacteria preparation: overnight LAB cultures were washed twice with PBS buffer, pH 7.2 before being resuspended in PBS at concentration of 2×10⁹ cfu/ml.

PMBC incubation: from these suspensions 10 μl was transferred into wells of the PMBC plates which were incubated at 37° C. in a 5% CO₂/95% air atmosphere. After 24 hours incubation the supernatant was aspirated, centrifuged at 2000 rpm and the supernatant removed and stored at −20° C. The control consisted of bacteria-free buffer.

Cytokine quantification: cytokine expression levels have been determined by ELISA tests (<<Enzyme linked immuno sorbent assay>>). ELISA plates are coated with anti-cytokine antibody (overnight procedure) and the antibody is blocked with PBS/BSA 1%. A proper standard was prepared with known concentrations of cytokines, covering the detection range of 15.62 to 2000 pg/ml (incubated overnight).

The anti-cytokine detection and quantification was performed with the streptavidine reaction on substrate (TMB Pharmigen). The commercial kits Pharmigen have been used according to the manufacturer's description. Four cytokines were determined: the pro-inflammatory/Th 1 cytokines TNFα, IFNγ, IL 12 and the anti-inflammatory/Th 2 cytokine IL10.

TABLE I

| | IL 10 | IL 12 | TNFα | IFNγ | IL10/IL12 |
|---|---|---|---|---|---|
| Control | 31.25 | 31.25 | 31.25 | 31.25 | 1 |
| KS 120.1 | 1228.67 | 176.32 | 17698.83 | 3513.36 | 6.96840971 |
| KS 121.1 | 2297.87 | 47.66 | 14180.66 | 897.65 | 48.2138061 |
| KS 116.1 | 2856.26 | 167.6 | 33569.91 | 7209.33 | 17.0540573 |
| KS 400 | 3177.49 | 103.85 | 26799 | 6949.13 | 30.5969186 |
| KS 300 | 2290.47 | 59.7 | 18703.66 | 10047.75 | 38.3663317 |
| KS 119.1 | 307.13 | 198.47 | 6693.3 | 9192.74 | 1.54748829 |
| KS 124.3 | 2969.02 | 660.98 | 31307.71 | 16985.56 | 4.49184544 |

### Observations

a high level of TNFα induction for all the tested LAB strains
a relatively low level of IFNγ concerning L. jensenii KS 121.1
the highest IL10 induction potential concerning L. crispatus KS 116.1 and KS 400
in contrast to the two L. jensenii strains the two L. gasseri strains have shown a similar profile, especially when considering the ratio's in IL10/IL12 and in TNFα/IFNγ.

Within the above testing frame it is clear that the cytokine induction profile is strain specific.

### Example 5

### Determination of the Anti-Inflammatory Activity

### In Vivo Test Using an Animal Model

An acute model of mice has been adapted from Camoglio et al. (see Eur. J. Immunol. 2000) where the animals have been fed from day −5 to day +2 with selected lactic acid bacteria strains, at a rate of 10⁸ bacteria per mouse per day. TNBS was then injected on day zero, at a rate of 120 mg/kg mice in order to induce acute colitis and the animals have been sacrificed at day +2 and eventually subjected to both macroscopic (Wallace score—Table I) and histological (Ameho score—Table II) scoring.

These tables clearly show that the selected lactic acid bacteria strains exhibit a significant anti-inflammatory effect when compared to reference strains.

### Example 6

### 6.1 Composition for Oral Administration

### Edible Capsules

Samples of the LAB strains of this invention (see above) have been cultured for min. 24 hours in conditions similar to those mentioned here above. The cultured strains have been isolated, washed and lyophilized individually, individually suspended in a lactose/MSK powder mixture and eventually divided into unit doses each of them containing about 10⁸-10⁹ cfu (colony forming units).

Edible cellulose capsules (hydroxypropyl methyl cellulose) each comprising about 10⁸-10⁹ cfu of selected LAB strains of this invention have been manufactured using a filler comprising the following ingredients:
dehydrated yoghurt powder
anhydrous dextrose
potato starch
microcrystalline cellulose
selected lyophilized LAB strain

### 6.2 Composition for Oral Administration

### Yoghurt

Portions of a so called “Yoghurt Nature Light” have been prepared using the following process: to a batch of standardized 1.5% fat milk there was added 3% of skimmed milk powder (MSK) and the whole was then pasteurized at 90° C. for 30 minutes. 1% volume of commercial starter cultures of L. bulgaricus and S. thermophilus have been added to the pasteurized milk; then the whole was gently stirred at room temperature, disposed in 100 ml containers which were all eventually incubated at 40° C. during around 4 hours to afford the desired pH.

Then portions of selected lyophilized LAB strains of this invention were added to the yoghurt cans in such an amount to have about 10⁸-10⁹ cfu per yoghurt can and a further incubation was carried out for about 30 min. until to afford a pH of about 4.5 to 4.7. These yoghurt portions can be stored at 4° C. before consumption.

TABLE II
TNBS colitis induced at day zero
Wallace score

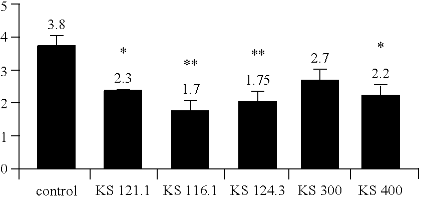

TABLE III
TNBS colitis induced at day zero
Ameho score

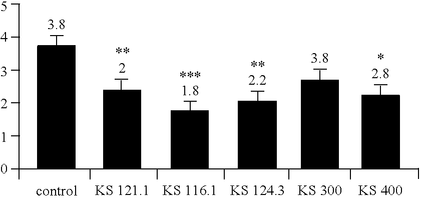

What is claimed is:

1. A method for treating a gastrointestinal infection in a subject, which comprises administering to a subject in need of such treatment a pharmaceutical preparation comprising a therapeutically effective amount of at least one strain selected from the group consisting of L. crispatus KS 116.1 (CNCM 1-3483), L. crispatus KS 119.4 (CNCM 1-3484), L. crispatus 127.1 (CNCM 1-3486), L. gasseri KS 114.1 (CNCM 1-3482), L. gasseri KS 120.1 (CNCM 1-3218), L. gasseri KS 123.1 (CNCM 1-3485), L. gasseri KS 124.3 (CNCM 1-3220), L. helveticus KS 300 (CNCM 1-3360), L. jensenii KS 119.1 (CNCM 1-32 17) and L. jensenii KS 121.1 (CNCM 1-3219) in combination with a pharmaceutically acceptable galenical carrier.

2. The method of claim 1 wherein the galenical carrier is designed for oral administration.

3. A method for inhibiting adhesion, colonization or growth of pathogens in the gastrointestinal tract of a subject, which comprises administering to a subject in need a pharmaceutical preparation comprising a therapeutically effective amount of at least one lactic acid bacteria strain selected from the group consisting of L. crispatus KS 116.1 (CNCM 1-3483), L. crispatus KS 119.4 (CNCM I-3484), L. crispatus 127.1 (CNCM 1-3486), L. gasseri KS 114.1 (CNCM 1-3482), L. gasseri KS 120.1 (CNCM 1-3218), L. gasseri KS 123.1 (CNCM 1-3485), L. gasseri KS 124.3 (CNCM 1-3220), L. helveticus KS 300 (CNCM 1-3360), L. jensenii KS 119.1 (CNCM 1-32 17) and L. jensenii KS 121.1 (CNCMI-3219) in combination with a pharmaceutically acceptable galenical carrier.

4. The method of claim 3 wherein the galenical carrier is designed for oral administration.

5. A method for modulating a cellular or humoral immune response at the vaginal or gastrointestinal level in a subject, which comprises administering to a subject in need thereof a pharmaceutical preparation comprising a therapeutically effective amount of at least one lactic acid bacteria strain selected from the group consisting of L. crispatus KS 116.1 (CNCM 1-3483), L. crispatus KS 119.4 (CNCM 1-3484), L. crispatus 127.1 (CNCM 1-3486), L. gasseri KS 114.1 (CNCM 1-3482), L. gasseri KS 120.1 (CNCM 1-3218), L. gasseri KS 123.1 (CNCM 1-3485), L. gasseri KS 124.3 (CNCM 1-3220), L. helveticus KS 300 (CNCM 1-3360), L. jensenii KS 119.1 (CNCM 1-3217) and L. jensenii KS 121.1 (CNCMI-3219 in combination with a pharmaceutically acceptable galenical carrier.

6. The method of claim 5, wherein the preparation is administered in an amount sufficient to inhibit an inflammatory or infectious syndrome.

7. The method of claim 5 wherein the galenical carrier is designed for oral administration.

8. A pharmaceutical composition comprising a therapeutically effective amount of at least one lactic acid bacteria strain selected from the group consisting of L. crispatus KS 116.1 (CNCM I-3483), L. crispatus KS 119.4 (CNCM 1-3484), L. crispatus 127.1 (NCM 1-3486), L. gasseri KS 114.1 (CNCM 1-3482), L. gasseri KS 120.1 (CNCM 1-32 18), L. gasseri KS 123.1 (CNCM 1-3485), L. gasseri KS 124.3 (CNCM 1-3220), L. helveticus KS 300 (CNCM 1-3360), L. jensenii KS 119.1 (CNCM 1-32 17) and L. jensenii KS 121.1 (CNCM 1-32 19) in combination with a pharmaceutically acceptable galenical carrier.

9. The composition of claim 8 wherein the galenical carrier is designed for oral administration.

10. The pharmaceutical composition of claim 9 which further comprises LAB growth factors.
